(19) United States
(12) Patent Application Publication
(10) Pub. No.: US 2026/0345082 A1
(43) Pub. Date: Nov. 5, 2026

(54) BED CONTROL DEVICE, BED DEVICE, MAGNETIC RESONANCE IMAGING APPARATUS, AND BED CONTROL METHOD

(71) Applicant: CANON KABUSHIKI KAISHA, Tokyo (JP)

(72) Inventors: Yutaka HOSHIYAMA, Tochigi (JP); Kagami FUJITA, Tochigi (JP); Yoshiteru WATANABE, Tochigi (JP)

(73) Assignee: CANON KABUSHIKI KAISHA, Tokyo (JP)

(21) Appl. No.: 19/662,355

(22) Filed: Apr. 29, 2026

(30) Foreign Application Priority Data
May 2, 2025 (JP) 2025-076706
Dec. 3, 2025 (JP) 2025-225453
Mar. 13, 2026 (JP) 2026-040827

(51) Int. Cl. A61B 5/00; A61B 5/055; G01R 33/30

(52) U.S. Cl. CPC A61B 5/704; A61B 5/055; G01R 33/307

(57) ABSTRACT

A bed control device of an embodiment includes processing circuitry. The processing circuitry calculates a position of a part of a subject to be imaged based on information regarding the part of the subject placed on a top plate provided in a bed device, which is to be imaged by a medical image diagnostic apparatus, and an image including the top plate and the subject placed on the top plate and captured by an imaging device, estimates a position of the top plate in a vertical direction, calculates an amount of movement of the top plate in a direction intersecting the vertical direction based on the calculated position of the part of the subject to be imaged and the estimated position in the vertical direction, and controls the movement of the top plate in the direction intersecting the vertical direction based on the amount of movement.

### CROSS-REFERENCE TO RELATED APPLICATION

The present application claims priority based on Japanese Patent Application No. 2025-076706 filed May 2, 2025, Japanese Patent Application No. 2025-225453 filed Dec. 3, 2025, and Japanese Patent Application No. 2026-040827 filed Mar. 13, 2026, the contents of which are incorporated herein by reference.

### FIELD

Embodiments disclosed in this specification and drawings relate to a bed control device, a bed device, a magnetic resonance imaging apparatus, and a bed control method.

### BACKGROUND

In a magnetic resonance imaging (MRI) apparatus, a technique of imaging a top plate before it enters a gantry using a 2D camera mounted on a ceiling and adjusting the amount (length) of the top plate that enters on the basis of the position of an imaging area of a subject and the position of the magnetic field center in the MRI apparatus has been known. With this configuration, the imaging area can be positioned at the magnetic field center for imaging, and degradation of image quality is reduced. However, in this case, imaging is performed with the top plate at its highest position, for example. Therefore, in a case in which the operator is short, and the like, for example, it may be difficult for the operator to perform subject setting works such as lifting the coil to the height of the top plate or wrapping the coil and fixing belt around the subject.

### BRIEF DESCRIPTION OF THE DRAWINGS

FIG. 1 is a diagram showing an example of a configuration of an MRI apparatus according to an embodiment.

FIG. 2 is a diagram showing an example of a configuration of fourth processing circuitry 20.

FIG. 3 is a flowchart showing an example of processing of the fourth processing circuitry 20.

FIG. 4 is a diagram showing an example of a bed image relative to the height of a top plate 34.

FIG. 5 is a simplified diagram showing the top plate 34 at a second height and a first height in a bed image.

FIG. 6 is a diagram showing an example of a configuration of fourth processing circuitry 50 in a second embodiment.

FIG. 7 is a diagram showing an example of a confidence level.

FIG. 8 is a diagram showing an example of skeletal information SK of a patient.

FIG. 9 is a flowchart showing an example of processing of the fourth processing circuitry 50 in the second embodiment.

FIG. 10 is a diagram showing an example of a configuration of fourth processing circuitry 60 in a third embodiment.

FIG. 11 is a diagram showing a series of flows until imaging by the MRI apparatus 100 is started.

### DETAILED DESCRIPTION

Hereafter, a bed control device, a bed device, a magnetic resonance imaging apparatus, and a bed control method of embodiments will be described with reference to the drawings.

A bed control device of an embodiment includes processing circuitry. The processing circuitry calculates a position of a part of a subject to be imaged based on information regarding the part of the subject placed on a top plate provided in a bed device, which is to be imaged by a medical image diagnostic apparatus, and an image including the top plate and the subject placed on the top plate and captured by an imaging device, estimates a position of the top plate in a vertical direction, calculates an amount of movement of the top plate in a direction intersecting the vertical direction based on the calculated position of the part of the subject to be imaged and the estimated position in the vertical direction, and controls the movement of the top plate in the direction intersecting the vertical direction based on the amount of movement.

### First Embodiment

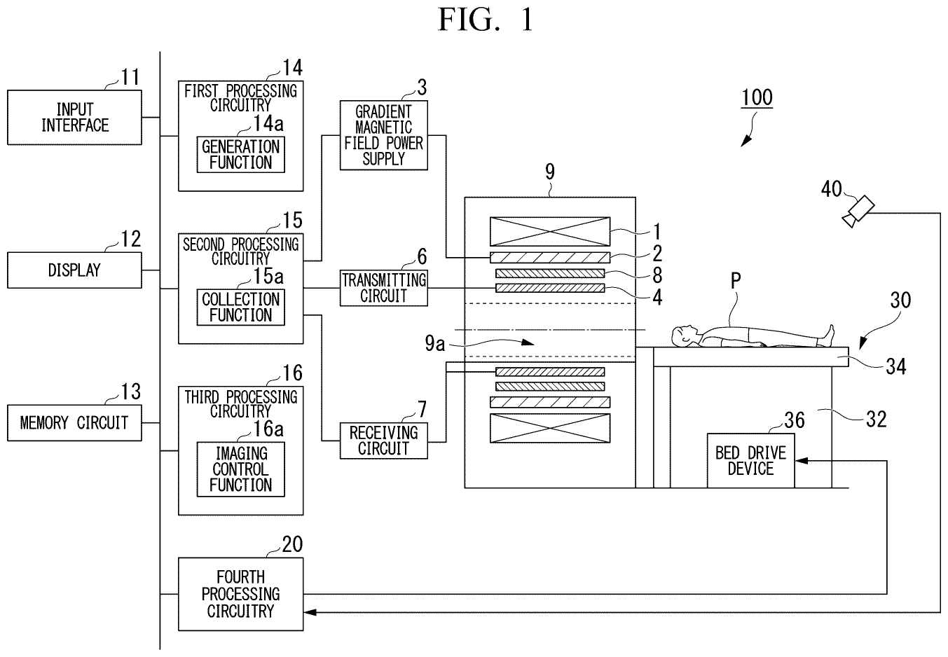

FIG. 1 is a diagram showing an example of a configuration of an MRI apparatus 100 according to a first embodiment. The MRI apparatus 100 is a medical imaging diagnostic apparatus installed in a room, such as an examination room. The MRI apparatus 100 includes, for example, a static magnetic field magnet 1, a gradient magnetic field coil 2, a gradient magnetic field power supply 3, a whole-body radio frequency (RF) coil 4, a transmitting circuit 6, a receiving circuit 7, an RF shield 8, a gantry 9, an input interface 11, a display 12, a memory circuit 13, first processing circuitry 14, second processing circuitry 15, third processing circuitry 16, fourth processing circuitry 20, a bed device 30, and a camera 40.

In the following description, a direction along a direction through which a bore 9a formed by the gantry 9 penetrates (the longitudinal direction of a top plate 34 in the bed device 30) is defined as the X direction, a direction perpendicular to the X direction in the horizontal plane is defined as the Y direction, and a direction perpendicular to both the X and Y directions (direction along the vertical direction) is defined as the Z direction.

The gradient magnetic field power supply 3 generates gradient magnetic fields within the bore 9a by supplying current to the gradient magnetic field coil 2. The gradient magnetic field power supply 3 generates gradient magnetic fields that change linearly in a readout direction, a phase encoding direction, and a slice direction which are orthogonal to each other by individually supplying current to the X coil, Y coil, and Z coil of the gradient magnetic field coil 2. The axis in the readout direction, the axis in the phase encoding direction, and the axis in the slice direction constitute a logical coordinate system for defining a slice region or a volume region to be imaged.

The gradient magnetic fields along the readout direction, phase encoding direction, and slice direction impart spatial position information to an NMR signal generated from a subject P. The gradient magnetic field in the readout direction imparts position information in the readout direction to the NMR signal.

The gradient magnetic field in the phase encoding direction imparts position information in the phase encoding direction to the NMR signal. The gradient magnetic field in the slice direction imparts position information in the slice direction to the NMR signal.

The whole-body RF coil 4 is disposed on the inner circumference side of the gradient magnetic field coil 2, applies RF pulses (excitation pulses, etc.) to the subject P placed in the bore 9a, and receives an NMR signal (echo signal, etc.) generated from the subject P due to the influence of the RF pulses. The whole-body RF coil 4 is formed in a hollow substantially cylindrical shape (including a shape with an elliptical cross-sectional shape perpendicular to the central axis).

The transmitting circuit 6 outputs an RF pulse signal corresponding to a resonance frequency (Larmor frequency) specific to a target atomic nucleus placed in the static magnetic field to the whole-body RF coil 4. The whole-body RF coil 4 applies RF pulses to the subject P located on the inner circumference side thereof on the basis of the RF pulse signal supplied from the transmitting circuit 6. The transmitting circuit 6 includes, for example, a pulse generator, an RF generator, a modulator, and an amplifier. The pulse generator generates the waveform of an RF pulse signal. The RF generator generates an RF signal at the resonance frequency.

The modulator generates an RF pulse signal by modulating the amplitude of an RF signal generated by the RF generator into a waveform generated by the pulse generator. The amplifier amplifies the RF pulse signal generated by the modulator. The amplifier outputs the amplified RF pulses to the whole-body RF coil 4, etc.

The whole-body RF coil 4 receives an NMR signal generated from the subject P due to the influence of the RF pulses and outputs the received NMR signal to the receiving circuit 7. For example, the whole-body RF coil 4 is a birdcage-type coil or a transverse electromagnetic (TEM) coil. The receiving circuit 7 generates NMR data based on the NMR signal output from the whole-body RF coil 4. The receiving circuit 7 outputs the generated NMR data to the second processing circuitry 15. The receiving circuit 7 includes, for example, a selector, a preamplifier, a phase detector, and an analog/digital (A/D) converter.

The selector selectively receives NMR signals output from the whole-body RF coil 4 and a local RF coil. The preamplifier amplifies the NMR signal output from the selector. The phase detector detects the phase of the NMR signal output from the preamplifier. The A/D converter generates NMR data by converting an analog signal output from the phase detector into a digital signal, and outputs the generated NMR data to the second processing circuitry 15. Processing described as being performed by the receiving circuit 7 does not necessarily have to be performed entirely by the receiving circuit 7, and some processing (for example, processing by the A/D converter) may be performed by the whole-body RF coil 4, etc.

The RF shield 8 is placed between the gradient magnetic field coil 2 and the whole-body RF coil 4. The RF shield 8 shields the gradient magnetic field coil 2 from the RF pulses generated by the whole-body RF coil 4. The RF shield 8 is formed in a hollow substantially cylindrical shape (including a shape with an elliptical cross-sectional shape perpendicular to the central axis of the cylinder) and is positioned in the space on the inner circumference side of the gradient magnetic field coil 2 to cover the outer circumference surface of the whole-body RF coil 4.

The gantry 9 has a hollow bore 9a formed in a substantially cylindrical shape (including a shape with an elliptical cross-sectional shape perpendicular to the central axis). The gantry 9 houses the static magnetic field magnet 1, the gradient magnetic field coil 2, the whole-body RF coil 4, and the RF shield 8. The gantry 9 houses the whole-body RF coil 4 on the outer circumference side of the bore 9a, the RF shield 8 on the outer circumference side of the whole-body RF coil 4, the gradient magnetic field coil 2 on the outer circumference side of the RF shield 8, and the static magnetic field magnet 1 on the outer circumference side of the gradient magnetic field coil 2. The space within the bore 9a of the gantry 9 becomes an imaging space where the subject P is positioned during imaging.

Although the MRI apparatus 100 of the first embodiment has a so-called tunnel-type structure in which the static magnetic field magnet 1, the gradient magnetic field coil 2, and the whole-body RF coil 4 are each formed in a substantially cylindrical shape, the MRI apparatus is not limited to those having a tunnel-type structure. For example, the MRI apparatus may have a so-called open-type structure in which a pair of static magnetic field magnets, a pair of gradient magnetic field coils, and a pair of RF coils are arranged facing each other across the imaging space in which the subject P is placed. In an open-type MRI apparatus, the space sandwiched between a pair of static magnetic field magnets, a pair of gradient magnetic field coils, and a pair of RF coils corresponds to the bore 9a in the tunnel-type structure.

The input interface 11 receives operations of inputting various instructions and various types of information from an operator. The operator is, for example, a technician or a doctor. The input interface 11 is connected to the first processing circuitry 14, the second processing circuitry 15, the third processing circuitry 16, and the fourth processing circuitry 20. The input interface 11 converts an input operation received from the operator into an electrical signal and outputs the electrical signal to the first processing circuitry 14, the second processing circuitry 15, the third processing circuitry 16, and the fourth processing circuitry 20.

The input interface 11 is realized by, for example, a trackball, a switch button, a mouse, and a keyboard for setting imaging conditions and a region of interest (ROI), a touchpad for input operations by touching an operating surface, a touchscreen having a display screen integrated with a touchpad, a non-contact input circuit using an optical sensor, and an audio input circuit.

In this specification, the input interface 11 is not limited to those equipped with physical operating components such as a mouse and a keyboard. For example, electrical signal processing circuitry that receives an electrical signal corresponding to an input operation from external input equipment provided separately from the apparatus and outputs the electrical signal to a control circuit is also included as an example of the input interface 11.

The display 12 displays various types of information. The display 12 is connected to the first processing circuitry 14, the second processing circuitry 15, the third processing circuitry 16, and the fourth processing circuitry 20. The display 12 converts data of various types of information sent from the first processing circuitry 14, the second processing circuitry 15, the third processing circuitry 16, and the fourth processing circuitry 20 into electrical signals for display and outputs the electrical signals. The display 12 is realized by, for example, a liquid crystal monitor, a CRT monitor, a touch panel, etc.

The memory circuit 13 stores various types of data. The memory circuit 13 is connected to the first processing circuitry 14, the second processing circuitry 15, the third processing circuitry 16, and the fourth processing circuitry 20. The memory circuit 13 stores various types of data input and output by each processing circuitry. The memory circuit 13 is realized by, for example, a semiconductor memory element such as a random access memory (RAM) or a flash memory, or by a hard disk, optical disc, or the like.

The first processing circuitry 14, the second processing circuitry 15, the third processing circuitry 16, and the fourth processing circuitry 20 realize such functions by, for example, a hardware processor executing a program stored in a memory device such as the memory circuit 13. The first processing circuitry 14, the second processing circuitry 15, the third processing circuitry 16, and the fourth processing circuitry 20 realize a processing function corresponding to each program by reading and executing each program from the memory circuit 13. In other words, each processing circuitry has the processing function shown in FIG. 1 in a state in which it has read each program.

The hardware processor refers to circuitry such as a central processing unit (CPU), a graphics processing unit (GPU), an application specific integrated circuit (ASIC), or a programmable logic device (e.g., a simple programmable logic device (SPLD), a complex programmable logic device (CPLD), or a field programmable gate array (FPGA)). Instead of storing a program in the memory circuit 13, the program may be directly embedded within the circuitry of the hardware processor. In this case, the hardware processor realizes functions by reading and executing the program embedded within the circuitry. The hardware processor is not limited to a single circuit and may be composed of a plurality of independent circuits combined to realize each function. Furthermore, a plurality of components may be integrated into a single hardware processor to realize each function.

The first processing circuitry 14, the second processing circuitry 15, the third processing circuitry 16, and the fourth processing circuitry 20 are each realized by a single processor, but the embodiment is not limited thereto. Each processing circuitry may be configured by combining a plurality of independent processors, and each processing function may be realized by each processor executing a program.

The processing functions of each processing circuitry may be realized by being appropriately distributed or integrated across one or more processing circuits. In the above description, a single memory circuit 13 is assumed to store the program corresponding to each processing function, but the embodiment is not limited thereto. For example, a plurality of memory circuits may be distributed and arranged for each processing circuitry, and each processing circuitry may read the corresponding program from the individual memory circuit.

The second processing circuitry 15 has a collection function 15a. The collection function 15a collects k-space data of the subject P on the basis of an imaging sequence output from the third processing circuitry 16. For example, the collection function 15a collects NMR data by driving the gradient magnetic field power supply 3, the transmitting circuit 6, the receiving circuit 7, and the local RF coil 5 according to various imaging sequences output from the third processing circuitry 16.

An imaging sequence is information that defines timing of supplying current from the gradient magnetic field power supply 3 to the gradient magnetic field coil 2 and the strength of the current, timing of supplying an RF pulse signal from the transmitting circuit 6 to the whole-body RF coil 4 and the strength of the RF pulse signal, timing of sampling an NMR signal by the receiving circuit 7, etc.

The collection function 15a stores NMR data output from the receiving circuit 7 and the local RF coil 5 in the memory circuit 13. The NMR data stored in the memory circuit 13 is stored as k-space data representing a two-dimensional or three-dimensional k-space, with position information in the readout direction, phase encoding direction, and slice direction imparted by the aforementioned gradient magnetic field.

The first processing circuitry 14 has a generation function 14a. The generation function 14a generates an MR image from the k-space data of the subject P collected by the collection function 15a of the second processing circuitry 15. The generation function 14a reads the k-space data collected by the collection function 15a of the second processing circuitry 15 from the memory circuit 13 and generates a two-dimensional or three-dimensional MR image by applying reconstruction processing such as a Fourier transform to the read-out k-space data. The generation function 14a stores the generated MR image in the memory circuit 13. The first processing circuitry 14 is an example of an MR image generation unit.

The third processing circuitry 16 has an imaging control function 16a. The imaging control function 16a receives input of imaging conditions from the operator via the input interface 11 and generates an imaging sequence for collecting k-space data of the subject P on the basis of the input imaging conditions. The imaging control function 16a outputs the generated imaging sequence to the second processing circuitry 15, causing the collection function 15a to collect k-space data.

The imaging control function 16a controls the third processing circuitry 16 to generate an MR image from the k-space data collected by the collection function 15a. In response to a request from the operator, the imaging control function 16a reads the MR image stored in the memory circuit 13 and displays the read MR image on the display 12.

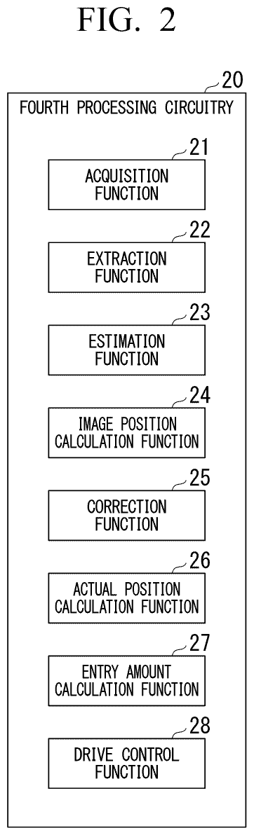

The fourth processing circuitry 20 controls, for example, the bed device 30. The fourth processing circuitry 20 is an example of a bed control device. FIG. 2 is a diagram showing an example of a configuration of the fourth processing circuitry 20. The fourth processing circuitry 20 includes, for example, an acquisition function 21, an extraction function 22, an estimation function 23, an image position calculation function 24, a correction function 25, an actual position calculation function 26, an entry amount calculation function 27, and a drive control function 28. Prior to describing the configuration of the fourth processing circuitry 20, the bed device 30 and the camera 40 will be described.

The bed device 30 includes, for example, a base 32, a top plate 34, and a bed drive device 36. The bed device 30 is installed, for example, such that the longitudinal direction of the top plate 34 is parallel to the central axis of the bore 9a of the gantry 9. The base 32 includes a housing that supports the top plate 34 such that the top plate 34 can move in the vertical direction (Y-axis direction). The top plate 34 is a plate-shaped member on which the subject P is placed.

The bed drive device 36 moves the top plate 34 on which the subject P is placed into the imaging space at the time of imaging the subject P. The bed drive device 36 includes a motor and an actuator. The bed drive device 36 moves the top plate 34 in the vertical direction and in the longitudinal direction (Z-axis direction) of the top plate 34.

For example, a switch for operating the bed drive device 36 is provided on the side of the base 32, or the like. When the operator operates the switch while the bed drive device 36 is stopped, the bed drive device 36 first raises the top plate 34 on which the subject P is placed to the upper limit, and then moves the top plate 34 into the bore 9a of the gantry 9, moving the top plate 34 to a position where a projector is at the center of the magnetic field.

The camera 40 is installed at a position in an examination room, for example, on the ceiling, where the camera 40 can image the bed device 30. The camera 40 captures an image (hereinafter referred to as a bed image) including the bed device 30, which has the top plate 34 on which the subject P is mounted, in the vicinity of the gantry 9. The camera 40 is, for example, an optical digital camera (optical camera). The camera 40 may also be an analog camera, and in this case, the camera 40 may have an A/D conversion function. The camera 40 may be any camera capable of imaging the subject P, such as an infrared camera. The camera 40 transmits the captured bed image to the fourth processing circuitry 20.

The acquisition function 21 in the fourth processing circuitry 20 acquires an image captured by the imaging device, including the subject mounted on the top plate of the bed device. The acquisition function 21 acquires a bed image, for example, by receiving the bed image transmitted by the camera 40. The acquisition function 21 is an example of an acquisition unit.

The extraction function 22 extracts the area occupied by the top plate 34 within the bed image (hereinafter referred to as a bed area). The extraction function 22 performs image processing on the bed image acquired by the acquisition function 21, for example. As a result of the image processing, the extraction function 22 extracts the top plate 34 included in the bed image. The extraction function 22 is an example of an extraction unit.

The estimation function 23 estimates the vertical position (hereinafter referred to as the depth) of the top plate 34. The estimation function 23 estimates the depth of the top plate 34, for example, on the basis of the bed area extracted by the extraction function 22. The estimation function 23 may estimate the depth of the top plate 34 by other methods. The estimation function 23 may estimate the depth of the top plate 34 on the basis of, for example, images from a separately provided depth camera, the control amount and detected movement amount of the bed drive device 36 (the amount of vertical movement of the top plate 34), the relationship between the height position of the bed device 30 captured from the side and a specific landmark, manual input by the operator, etc. The estimation function 23 is an example of an estimation unit.

The image position calculation function 24 calculates information regarding a part of the subject P placed on the top plate 34 provided on the bed device 30, which is to be imaged by the MRI apparatus 100 (hereinafter referred to as an imaged area), for example, a specific position in the image. Specifically, the image position calculation function 24 calculates the imaged area in the bed image when the top plate 34 is located at a first height in the vertical direction as a specific position in the image on the basis of information indicating the imaged area of the subject P set by the operator through the input interface 11. For example, if the imaged area set by the operator is the head, the image position calculation function 24 processes the bed image to extract the head of the subject P within the bed image. On the basis of the positional relationship between the extracted head of the subject P and the bed area extracted by the extraction function 22, the image position calculation function 24 determines the position of the imaged area of the subject P on the top plate 34. The first height corresponds, for example, to the height of the top plate 34 that makes it easy for the operator to work on the subject P mounted on the top plate 34. The image position calculation function 24 is an example of a first calculation unit.

The correction function 25 corrects the specific position in the image calculated by the image position calculation function 24 on the basis of the first height and a second height. Specifically, the correction function 25 corrects the specific position in the image calculated by the image position calculation function 24 on the basis of, for example, the difference between the first height and the second height. The difference between the first height and the second height corresponds to the depth estimated by the estimation function 23. The correction function 25 calculates the position of the imaged area of the subject P after correction on the basis of, for example, the position of the imaged area of the subject P before correction and a predetermined calculation formula. The correction function 25 is an example of a correction unit. The second height corresponds to, for example, the vertical position (height) when the top plate 34 is inserted into the bore 9a.

The actual position calculation function 26 calculates the position in the real space (real-space specific position) corresponding to the specific position in the image on the basis of the specific position in the image corrected by the correction function 25. The actual position calculation function 26 calculates the relative position of the imaged area of the subject P with respect to the top plate 34 in the real space as the real-space specific position, for example, by using a predetermined calculation formula based on the specific corrected position in the image corrected by the correction function 25.

The entry amount calculation function 27 calculates the amount of movement of the top plate 34 in the longitudinal direction (X direction) when the top plate 34 enters the bore 9a (hereinafter referred to as an entry amount) on the basis of the real-space specific position calculated by the correction function 25. The entry amount calculation function 27 determines the entry amount when the top plate 34 enters the bore 9a on the basis of the distance in the X direction between the real-space specific position calculated by the actual position calculation function 26 and a predetermined position in an imaging area within the imaging space of the bore 9a (an area where the static magnetic field generated by the static magnetic field magnet 1 is relatively uniform and imaging of the imaged area of the subject P is performed), for example, a reference position (for example, magnetic field center position). The reference position is the position in the vertical direction when the top plate 34 is inserted into the bore 9a in the gantry 9 of the MRI apparatus 100. The entry amount calculation function 27 is an example of a second calculation unit.

The entry amount calculation function 27 calculates the amount of movement of the top plate 34 in the longitudinal direction (X direction) intersecting the vertical direction (Z direction) on the basis of the position of the top plate 34 in the vertical direction at the timing at which the image used by the image position calculation function to calculate the position of the imaged area of the subject P has been captured by the camera 40, for example.

The drive control function 28 controls the movement of the top plate 34 in the longitudinal direction (X direction) of the top plate 34 on the basis of the amount of movement (entry amount) calculated by the entry amount calculation function 27. The drive control function 28 controls the movement of the top plate 34 by transmitting a drive control signal to the bed drive device 36, for example. The drive control function 28 is an example of a drive control unit.

Next, processing of the MRI apparatus 100 in the first embodiment will be described. In the MRI apparatus 100, at the time of examining the subject P, the operator places the subject P on the top plate 34 and performs work such as attaching an RF coil to the subject P placed on the top plate 34. When performing such work, the operator adjusts the height of the top plate 34 such that the top plate 34 is at a height (first height) that allows the operator to easily perform the work on the top plate 34 with respect to the subject P in consideration of the height of the operator, and the like. The height of the top plate 34 is adjusted by the bed drive device 36, for example, by the operator instructing the adjustment of the height of the top plate 34 via the input interface 11.

The operator performs work on the subject P with the top plate 34 at the first height. After completing the work on the subject P, the operator moves the top plate 34 to the second height, and then inserts the top plate 34 into the bore 9a of the gantry 9. The operations of moving the top plate 34 to the second height and moving the top plate 34 into the bore 9a are performed by the bed drive device 36, for example, by the operator giving instructions via the input interface 11.

When performing work on the subject P before the top plate 34 enters the bore 9a of the gantry 9, once the height of the top plate 34 has been adjusted, the camera 40 captures a bed image including the subject P mounted on the top plate 34 according to operation of the operator. The camera 40 transmits the captured bed image to the fourth processing circuitry 20.

The memory circuit 13 stores, for example, the distance from the camera 40 to the top plate 34 in the height direction when the top plate 34 is positioned at the second height, for example, the highest position (hereinafter referred to as a reference depth), and the size (area) of the bed area in the bed image obtained by the camera 40 capturing the top plate 34 positioned at the second height.

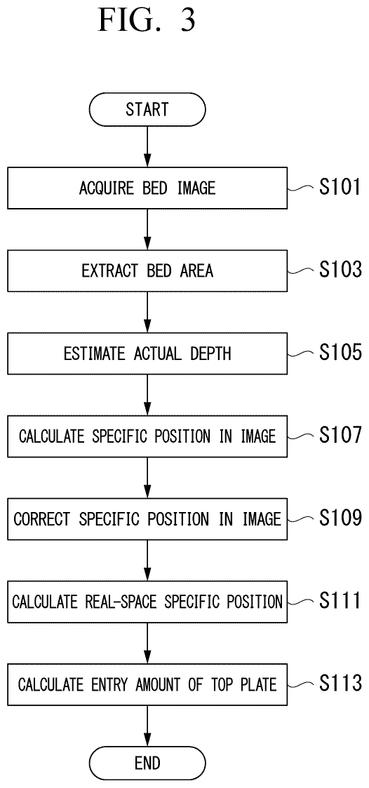

After such preparations are completed, the following processing is performed in the fourth processing circuitry 20. FIG. 3 is a flowchart showing an example of the processing in the fourth processing circuitry 20. The fourth processing circuitry 20 first acquires the bed image transmitted by the camera 40 using the acquisition function 21 (step S101). Next, the extraction function 22 processes the bed image acquired by the acquisition function 21 to identify the top plate 34 included in the bed image and extracts the bed area occupied by the top plate 34 (step S103).

Next, the estimation function 23 reads the size (area) of the bed area extracted by the extraction function 22 (hereinafter, a target bed area) and the size (area) of the bed area when the top plate 34 is positioned at the second height position stored in the memory circuit 13 (hereinafter, a reference bed area). On the basis of a result of comparison between the size (area) of the target bed area and the size (area) of the reference bed area, and the reference depth, the estimation function 23 estimates the distance from the camera 40 to the top plate 34 in the height direction (hereinafter, an actual depth) (step S105).

Next, the image position calculation function 24 calculates a specific position in the bed image (step S107). The specific position in the image is identified, for example, on the basis of image processing results of the bed image in the image position calculation function 24. The specific position in the image is identified, for example, by the number of px increasing towards the right side in the X direction when the end of the gantry 9 is set as 0 px.

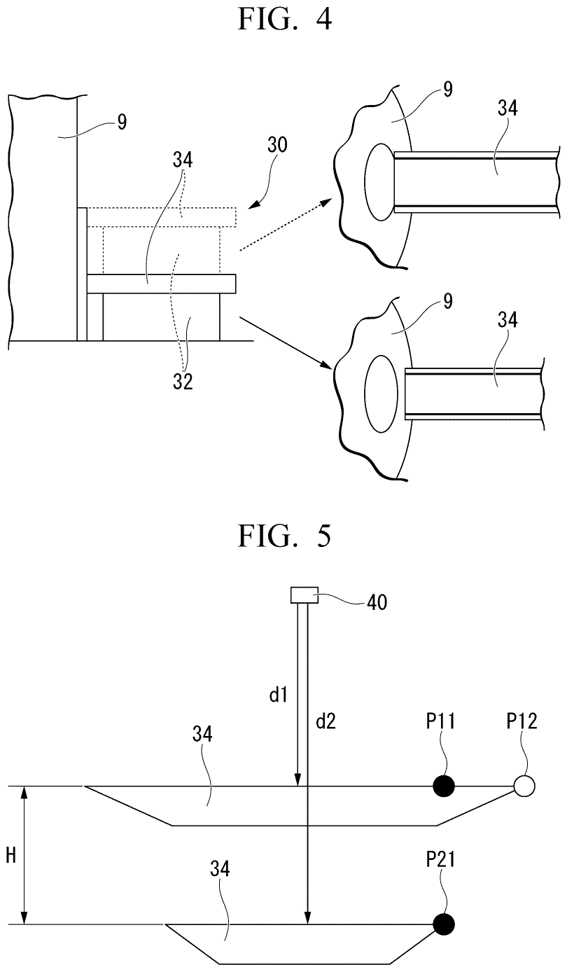

Next, the correction function 25 corrects the specific position in the image (step S109). The relationship between the case in which the top plate 34 in the bed image is at the second height and the case in which the top plate 34 is at the first height will be described. FIG. 4 is a diagram showing an example of a bed image with respect to the height of the top plate 34. In the left diagram of FIG. 4, a state in which the top plate 34 is at the second height is indicated by a dashed line, and a state in which the top plate 34 is at the first height is indicated by a solid line. An example of a bed image when the top plate 34 is at the second height is shown in the upper right of FIG. 4, and an example of a bed image when the top plate 34 is at the first height is shown in the lower right of FIG. 4.

As can be ascertained from comparison between the upper right and lower right of FIG. 4, the top plate 34 at the second height appears larger in the bed image than the top plate 34 at the first height. Therefore, even if a specific position in the image is calculated when the subject P is placed on the top plate 34 at the first height, the specific position in the image will be shifted when the top plate 34 is at the second height.

FIG. 5 is a simplified diagram showing the top plate 34 at the second height and the first height in the bed image. In the bed image, when the top plate 34 is at the first height, a 100px position P21 is the projector position. On the other hand, when the top plate 34 is at the second height, the 100px position P11 is not the projector position, and a 120px position P12 becomes the specific position in the image. The correction function 25 corrects the projector position calculated by the image position calculation function 24, for example, the 100px position P21 shown in FIG. 5, to the 100px position P11.

The correction function 25 corrects the specific position in the image using, for example, the following formula (1). The coefficients α, β, and γ in the formula (1) are calculated and acquired in advance and stored, for example, in the memory circuit 13.

x′=x+α×(x−W/2)/{β×H+γ}×H (1)

Here, x: Specific position in the image before correction (px)
x′: Specific position in the image after correction (px)
W: Size of the top plate 34 in the head-to-tail direction in the bed image
H: d2−d1
d1: Distance from the camera 40 to the top plate 34 at the second height
d2: Distance from the camera 40 to the top plate 34 at the first height
α: Degree of change of each position in the image due to change in the height of the top plate 34
β: Degree of change in the interval between positions in the image due to the change in the height of the top plate 34
Γ: Intercept

Next, the actual position calculation function 26 calculates a real-space specific position on the basis of the result of correction by the correction function 25 (step S111). Next, the entry amount calculation function 27 calculates the entry amount of the top plate 34 on the basis of the real-space specific position calculated by the actual position calculation function 26 (step S113).

In calculating the entry amount of the top plate 34, for example, the entry amount calculation function 27 acquires an imaged area on the basis of an image at the timing when the operator performs an input operation on the input interface 11, and calculates the height of the top plate 34 at that timing, after the subject P is placed on the top plate 34 and before a blanket and coil are set on the subject P. Alternatively, moving images of the bed device 30 and the subject P are continuously captured, and the entry amount calculation function 27 extracts the imaged area on the basis of the video at the timing immediately before the blanket and coil are set on the subject P, and calculates the height of the top plate 34 at that timing.

In this manner, the fourth processing circuitry 20 completes the processing shown in FIG. 3. After the fourth processing circuitry 20 completes the processing shown in FIG. 3, the drive control function 28 drives the bed drive device 36 to move the top plate 34 to the second height position, and moves the top plate 34 into the bore 9a of the gantry 9 by the entry amount calculated by the entry amount calculation function 27. Thereafter, examination using the MRI apparatus 100 is started.

The MRI apparatus 100 of the first embodiment calculates the real-space specific position on the basis of the specific position in the image and the depth of the top plate 34. Therefore, since the real-space specific position can be calculated with the top plate 34 positioned at a work position, it is possible to reduce the degradation of image quality while making it easier for the operator to work.

### Second Embodiment

Next, a second embodiment will be described. The MRI apparatus 100 of the second embodiment differs from the first embodiment mainly with respect to the configuration and processing of the fourth processing circuitry. The MRI apparatus 100 of the second embodiment will be described below, focusing on the differences from the first embodiment. In the following description, components having the same functions as those of the first embodiment are denoted by the same reference numerals, and descriptions may be omitted.

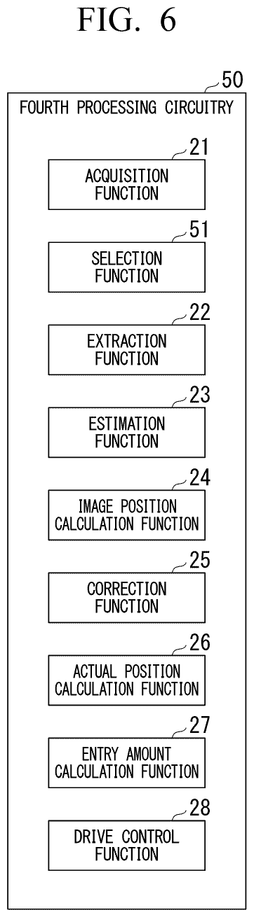

FIG. 6 is a diagram showing an example of the configuration of the fourth processing circuitry 50 of the second embodiment. The fourth processing circuitry 50 includes, for example, an acquisition function 21, a selection function 51, an extraction function 22, an estimation function 23, an image position calculation function 24, a correction function 25, an actual position calculation function 26, an entry amount calculation function 27, and a drive control function 28. The acquisition function 21, the extraction function 22, the estimation function 23, the image position calculation function 24, the correction function 25, the actual position calculation function 26, the entry amount calculation function 27, and the drive control function 28 have the same functions as those in the first embodiment.

The camera 40 captures a plurality of images (hereinafter referred to as “original images”) in the examination room at different timings. The original images include a bed image. For example, the camera 40 starts capturing images of the examination room after a subject or an operator enters the examination room. The camera 40 may capture images of the examination room for a predetermined period of time, for example, during hospital hours, or continuously for 24 hours.

The camera 40 may be a camera that captures moving images (video) instead of images. The camera 40 transmits the plurality of original images, including the captured bed image, to the fourth processing circuitry 50. The fourth processing circuitry 50 stores the transmitted original images in the memory circuit 13. Such processing may also be performed in the first embodiment.

The selection function 51 in the fourth processing circuitry 50 selects support images from among the plurality of original images captured by the camera 40 at different timings on the basis of a confidence level set for each original image. The support images are images used to assist work in the MRI apparatus 100, and include, for example, a bed image and images used to calculate the projector position as shown in the first embodiment. The selection function 51 selects support images from among the plurality of original images stored in the memory circuit 13.

The selection function 51 calculates the confidence level for each of the plurality of original images stored in the memory circuit 13, for example. The confidence level is, for example, a confidence level for detecting or identifying a target object. For example, in a case in which the projector position is identified on the basis of images, the confidence level is the probability that the projector position can be identified, and an image with a higher confidence level makes it easier to identify the projector position.

The confidence level is calculated on the basis of, for example, the positional relationship between the subject and the bed device 30 (top plate 34), and the success or failure of detecting the landmark set on the subject, and the like. The confidence level is, for example, a confidence level regarding the projector position. In this case, the landmark is, for example, a mark corresponding to a specific position on the subject used to determine the projector position. For example, an image in which a landmark is captured therein, or the position of the landmark can be identified will have a high confidence level. Alternatively, an image in which the relative positional relationship between the subject and the top plate 34 can be identified with high accuracy will have a higher confidence level.

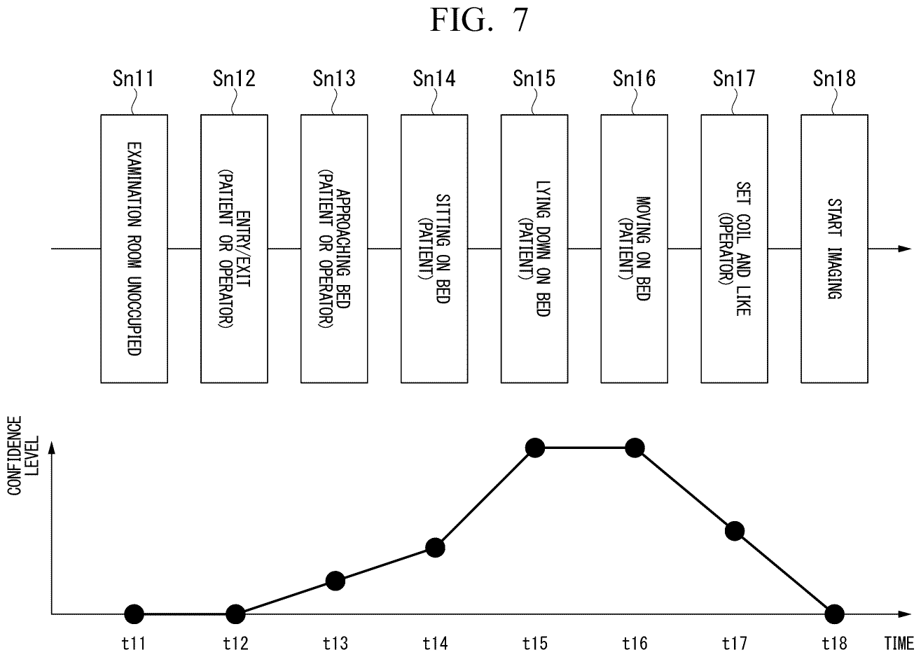

The selection function 51 calculates the confidence level on the basis of, for example, the situation in the examination room. FIG. 7 is a diagram showing an example of confidence levels. FIG. 7 shows a series of flows in the examination room from an unoccupied state to the entry of a patient (subject) or operator and the start of imaging by the MRI apparatus 100, and the confidence level at each scene at each timing within the flows.

The following first scene Sn11 to eighth Sn18 are assumed. In the first scene Sn11, the examination room is unoccupied. A patient or operator enters the examination room in the following second scene Sn12, the patient or operator approaches the bed device 30 in the third scene Sn13, and the patient sits on the top plate 34 of the bed device 30 in the fourth scene Sn14.

Then, the patient lies down on the top plate 34 in the fifth scene Sn15. In the following sixth scene Sn16, the patient moves on the top plate 34 such that the operator can easily perform settings, etc., and in the seventh scene Sn17, the operator sets coils, etc. After the setting is complete, the top plate 34 is caused to enter the gantry 9 and imaging of the patient is started in the eighth scene Sn18.

In the process up to this point, in the first scene Sn11 and the second scene Sn12, the patient has not reached the vicinity of the bed device 30. In this state, it is not possible to capture a person (patient) on (including the vicinity of) the top plate 34, and thus the selection function 51 calculates the first confidence level t11 of the image in the first scene Sn11 and the second confidence level t12 of the image in the second scene Sn12 as “none (=0).”

Subsequently, in the third scene Sn13, the patient is approaching the bed. Even in this state, it is not possible to capture the patient on the top plate 34, and thus the patient is not as close to the bed device as in the first scene Sn11 and the second scene Sn12. Therefore, the selection function 51 calculates the third confidence level t13 of the image in the third scene Sn13 as “low,” which is higher than “none (=0).”

In the following fourth scene Sn14, the patient is sitting on the top plate 34. In this state, the patient can be captured on the top plate 34, but the patient is not in a stable state to be seen lying down on the top plate 34. Therefore, the selection function 51 calculates the fourth confidence level t14 of the image in the fourth scene Sn14, as “medium,” which is higher than the third confidence level t13.

In the following fifth scene Sn15, the patient is lying on the top plate 34. Furthermore, in the sixth scene Sn16, the position of the patient lying on the top plate 34 is adjusted and moved by the operator, and then enters a stopped state. In this state, the patient's body is on the top plate 34. Furthermore, if a landmark can be detected, the confidence level will be even higher. Assuming that a landmark can be detected, the selection function 51 calculates the fifth confidence level t15 of the image in the fifth scene Sn15 and the sixth confidence level t16 of the image in the sixth scene Sn16 as “highest.”

In the following seventh scene Sn17, the operator has set the coils, etc., on the patient. In this state, although the patient's body is on the top plate 34, a landmark may not be detectable due to the coils, a blanket, etc. In this case, the selection function 51 calculates the seventh confidence level t17 of the seventh scene Sn17 as “high,” which is higher than “medium” but lower than “highest.”

For example, the selection function 51 processes the original images to determine whether a scene of the original images is one of the first scene Sn11 to the seventh scene Sn17. After determining the scene of the original images, the selection function 51 calculates the confidence level corresponding to the determined scene and selects the original image with the highest confidence level as a support image.

If there is a plurality of original images with the highest confidence level, the selection function 51 selects the most recent original image over time as a support image. For example, if there are images of the fifth scene Sn15 and the sixth scene Sn16 with the confidence level of “highest,” the selection function 51 selects the image of the most recent scene, in this case, the image of the sixth scene Sn16, as a support image. The selection function 51 is an example of a selection unit.

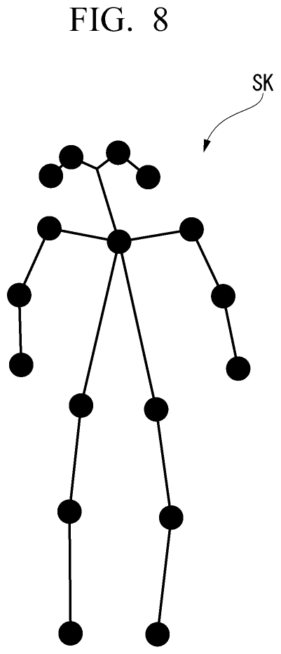

The selection function 51 may calculate confidence levels using other factors in addition to or instead of the situation in the examination room. For example, the selection function 51 may model a patient, perform skeletal detection at a plurality of points on the patient, and calculate confidence levels on the basis of the results of the skeletal detection. FIG. 8 shows an example of skeletal information SK of a patient.

The skeletal information SK holds information such as predetermined feature points, such as joints, the distance between feature points, and the angles between skeletal structures connecting feature points. The selection function 51 may calculate predetermined feature points of the patient from images captured by the camera 40, calculate the projector position from this skeletal information of a plurality of points, and calculate a confidence level from the calculated projector position.

The selection function 51 may determine each scene without calculating confidence levels and select images from scenes where confidence levels are expected to be high, for example, the fifth scene Sn15 and the sixth scene Sn16, as support images. If a plurality of support images are selected, the support images may be selected by calculating numerical values of the confidence levels.

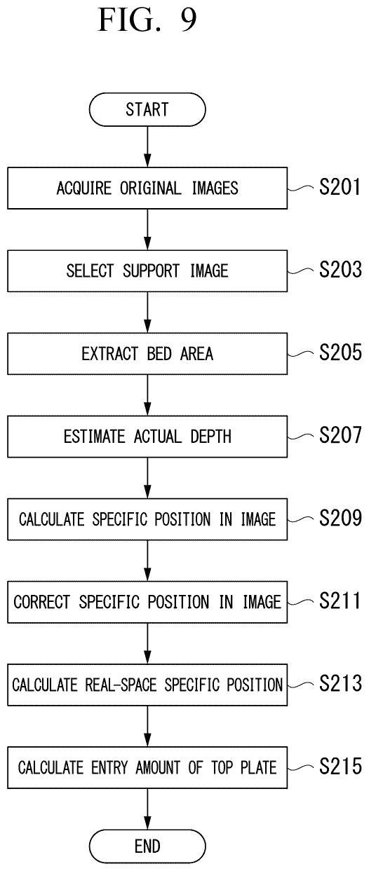

Next, processing of the fourth processing circuitry 50 of the second embodiment will be described. FIG. 9 is a flowchart showing an example of the processing of the fourth processing circuitry 50 of the second embodiment. In the fourth processing circuitry 50 of the second embodiment, first, the acquisition function 21 acquires an original image transmitted by the camera 40 (step S201).

The acquisition function 21 stores the acquired original image in the memory circuit 13. The acquisition function 21 stores a plurality of original images in the memory circuit 13. Subsequently, the selection function 51 calculates confidence levels of the plurality of original images stored in the memory circuit 13, and selects a support image from the plurality of original images on the basis of the calculated confidence levels (step S203).

Thereafter, as in the first embodiment, the fourth processing circuitry 50 extracts a bed area occupied by the top plate 34 using the extraction function 22 (step S205), and estimates the actual depth using the estimation function 23 (step S207). Furthermore, the fourth processing circuitry 50 calculates a specific position in the image using the image position calculation function 24 (step S209), and corrects the specific position in the image using the correction function 25 (step S211). Subsequently, the fourth processing circuitry 50 calculates a real-space specific position using the actual position calculation function 26 (step S213), and calculates the entry amount of the top plate 34 using the entry amount calculation function 27 (step S215). In this manner, the fourth processing circuitry 50 completes the processing shown in FIG. 9.

While the fourth processing circuitry 50 is calculating the entry amount of the top plate 34, the operator performs settings on the patient on the top plate 34. When the operator has completed the settings, imaging by the MRI apparatus 100 is started. When imaging by the MRI apparatus 100 is started, the operator operates a switch located on the side of the base 32. In response to the switch operation, the bed drive device 36 raises the top plate 34 to the upper limit, and then moves the top plate 34 to a position where the projector position is at the center of the magnetic field.

The MRI apparatus 100 of the second embodiment provides the same effects as the first embodiment. On the other hand, in conventional MRI apparatuses, there is a technique of setting the projector position on a subject and adjusting the entry amount (length) of the top plate on the basis of the projector position and the position of the magnetic field center in the MRI apparatus. In setting the projector position, the subject mounted on the top plate before entering the gantry is imaged by a 2D camera installed on the ceiling to acquire an image, and the imaging position of the subject is detected on the basis of the acquired image. The projector position is set on the basis of the detected imaging position. By setting the projector position, the subject position can be precisely determined, thereby reducing discomfort to the subject caused by laser light projection and minimizing operator errors.

On the other hand, the operator performs settings for the subject, such as wrapping coils and a securing belt around the subject on the top plate. During the settings, the operator lifts the coils to the height of the top plate and wraps the coils and securing belt around the subject. Therefore, if the top plate is high relative to the height of the operator, the operator may have difficulty performing settings for the subject. Thus, it is conceivable to adjust the height position of the top plate to a position that is easy for the operator to operate depending on the height of the operator, or the like.

Imaging of the subject by the 2D camera mounted on the top plate is performed, for example, after the operator has performed settings for the subject, but if the imaged area at the time of imaging the subject by the 2D camera is hidden by the coils or blanket, it is difficult to detect the imaged area. Therefore, the operator may need to determine the appropriate timing for detection and initiate device detection, which can hinder smooth settings for the subject.

On the other hand, the MRI apparatus 100 of the second embodiment selects a support image on the basis of the confidence level set for each original image using the selection function 51. Therefore, the projector position can be set on the basis of the original images captured by the camera 40 in an appropriate scene, allowing for smooth settings for the subject.

### Third Embodiment

Next, a third embodiment will be described. The MRI apparatus 100 of the third embodiment differs from the first and second embodiments mainly with respect to the configuration and processing of the fourth processing circuitry. The MRI apparatus 100 of the third embodiment will be described below, focusing on the differences from the first and second embodiments.

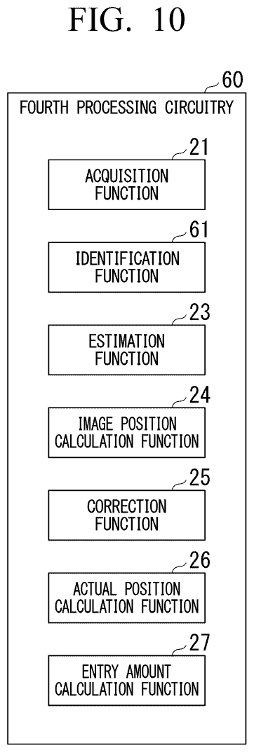

FIG. 10 is a diagram showing an example of the configuration of the fourth processing circuitry 60 in the third embodiment. The fourth processing circuitry 60 includes, for example, an acquisition function 21, an identification function 61, an extraction function 22, an estimation function 23, an image position calculation function 24, a correction function 25, an actual position calculation function 26, an entry amount calculation function 27, and a drive control function 28. The acquisition function 21, the extraction function 22, the estimation function 23, the image position calculation function 24, the correction function 25, the actual position calculation function 26, the entry amount calculation function 27, and the drive control function 28 have the same functions as in the first embodiment.

The camera 40 captures a moving image in the examination room. The camera 40 transmits the captured moving image to the fourth processing circuitry 60. The fourth processing circuitry 60 stores the transmitted moving image in the memory circuit 13. The camera 40 captures image scenes as moving images in a continuous sequence during hospital hours or for a predetermined period of time, such as 24 hours.

The identification function 61 in the fourth processing circuitry 60 performs scene analysis for analyzing the captured image scenes of the moving images stored in the memory circuit 13. On the basis of the results of the scene analysis, the identification function 61 acquires a detectable timing. The identification function 61 identifies a frame image extracted at the acquired detectable timing in the moving images, for example, as a support image.

A detectable timing is a timing at which a detection target, for example, the projector position, becomes detectable. A detectable timing is, for example, a timing at which the confidence level is high, for example, the highest. The identification function 61 sets, for example, a state in which the patient is lying on the top plate or a state in which the patient is moving on the top plate as a candidate for the detectable timing.

The identification function 61 identifies the most recent candidate for the detectable timing as the detectable timing when there is a plurality of candidates for the detectable timing. The identification function 61 identifies a frame image corresponding to a frame in the moving image at the identified detectable timing, for example, as a support image. The identification function 61 is an example of an identification unit.

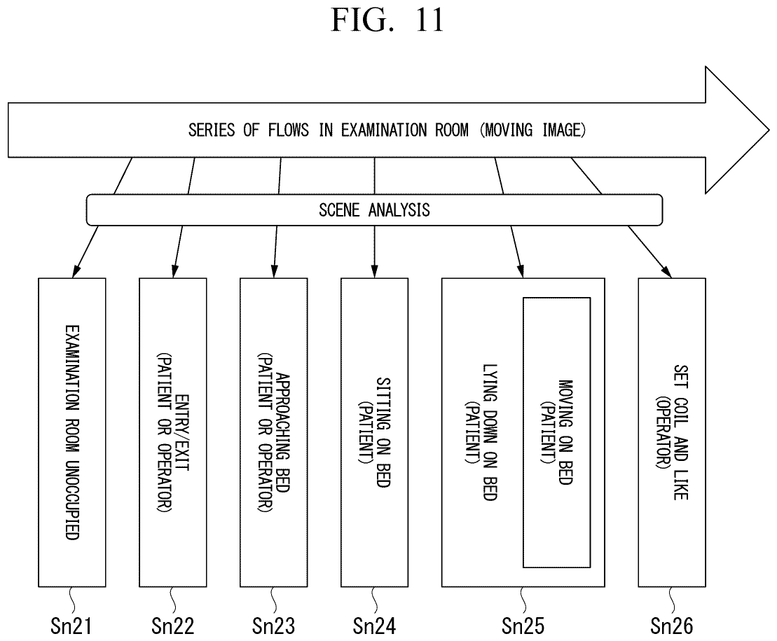

FIG. 11 is a diagram showing a series of flows until imaging by the MRI apparatus 100 is started. FIG. 11 shows a series of flows in the examination room from an unoccupied state until a patient (subject) or operator enters the room and imaging by the MRI apparatus 100 is started. The camera 40 captures this series of situations in the examination room as a moving image.

The following first scene Sn21 to sixth scene Sn26 are assumed. In the first scene Sn21, the examination room is unoccupied. A patient or operator enters the examination room in the following second scene Sn22, the patient or operator approaches the bed device 30 in the third scene Sn23, and the patient sits on the top plate 34 of the bed device 30 in the fourth scene Sn24.

Thereafter, in the fifth scene Sn25, the patient lies down on the top plate 34 and then moves on the top plate 34 such that the operator easily performs settings, etc. In the following sixth scene Sn26, the operator sets the coils, etc. After the setting is complete, the top plate 34 is caused to enter the gantry 9, and imaging of the patient is started.

The identification function 61 identifies a scene with a high confidence level from among the scenes. Specifically, the identification function 61 identifies the fifth scene Sn25 in which the patient is lying on the top plate 34 or moving on the top plate 34. Through scene analysis, the identification function 61 detects a detectable timing at which the fifth scene Sn25 ends, and identifies a frame image extracted at the detected detectable timing as a support image.

The MRI apparatus 100 of the third embodiment provides the same effects as the first embodiment. Furthermore, the MRI apparatus 100 of the third embodiment performs scene analysis using the identification function 61 and acquires a detectable timing on the basis of the results of the scene analysis. The identification function 61 identifies the frame image extracted in the frame at the acquired detectable timing in the moving image as a support image. Therefore, it is possible to set the projector position, etc. on the basis of a moving image captured by the camera 40 in an appropriate scene. Accordingly, the subject can be set smoothly.

In the above embodiment, the bed image is processed to directly extract the bed area, but a member installed at a fixed position relative to the bed area, such as a head coil, may be extracted and the bed area may be extracted on the basis of the position of the head coil. Alternatively, an area that is easy to extract may be removed from the entire bed image, and the remaining area may be defined as a bed area, or an area with a distance difference may be defined as a bed area. Examples of areas that are easy to extract from the entire bed image include the gantry 9, the head coil, the subject P, the floor surface on which the MRI apparatus 100 is installed, etc. In the above embodiment, the bed device is provided on the MRI apparatus 100, but it may also be a medical imaging diagnostic apparatus other than the MRI apparatus 100, such as an X-ray CT apparatus or an X-ray TV apparatus. In the above embodiment, a real-space specific position is calculated, and the entry amount of the top plate 34 is calculated on the basis of the real-space specific position, but the calculation of the real-space specific position may be omitted, and the entry amount of the top plate 34 may be calculated using a specific position in the image before and after correction.

According to at least one embodiment described above, a bed control device can reduce the degradation of image quality while making it easier for the operator to work by including a first calculation unit that calculates the position of a part of a subject to be imaged on the basis of information regarding the part of the subject placed on the top plate provided on the bed device, which is to be imaged by a medical image diagnostic apparatus, and an image including the top plate and the subject placed on the top plate and captured by an imaging device, an estimation unit that estimates a position of the top plate in the vertical direction, a second calculation unit that calculates the amount of movement of the top plate in a direction intersecting the vertical direction on the basis of the position calculated by the first calculation unit and the position estimated by the estimation unit, and a drive control unit that controls the movement of the top plate in the direction intersecting the vertical direction on the basis of the amount of movement calculated by the second calculation unit.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A bed control device comprising processing circuitry configured to:
calculate a position of a part of a subject to be imaged based on information regarding the part of the subject placed on a top plate provided in a bed device, which is to be imaged by a medical image diagnostic apparatus, and an image including the top plate and the subject placed on the top plate and captured by an imaging device;
estimate a position of the top plate in a vertical direction;
calculate an amount of movement of the top plate in a direction intersecting the vertical direction based on the calculated position of the part of the subject to be imaged and the estimated position in the vertical direction; and
control the movement of the top plate in the direction intersecting the vertical direction based on the calculated amount of movement.

2. The bed control device according to claim 1, wherein the processing circuitry is configured to calculate the amount of movement of the top plate in the direction intersecting the vertical direction based on the position of the top plate in the vertical direction at a timing at which the image used to calculate the position of the part of the subject to be imaged has been captured by the imaging device.

3. The bed control device according to claim 1, wherein the processing circuitry is further configured to correct the calculated position of the part of the subject to be imaged based on a distance between the estimated position in the vertical direction and a reference position in the vertical direction, and to calculate the amount of movement of the top plate in the direction intersecting the vertical direction based on the corrected position of the part of the subject to be imaged.

4. The bed control device according to claim 3, wherein the reference position is a position in the vertical direction when the top plate is inserted into a bore of the medical image diagnostic apparatus.

5. The bed control device according to claim 3, wherein the processing circuitry is configured to calculate the amount of movement of the top plate in the direction intersecting the vertical direction based on a distance between the corrected position of the part of the subject to be imaged in the direction intersecting the vertical direction and a predetermined position in the direction intersecting the vertical direction.

6. The bed control device according to claim 5, wherein the predetermined position in the direction intersecting the vertical direction is a position within an imaging space in a bore of the medical image diagnostic apparatus which is a magnetic resonance imaging apparatus.

7. The bed control device according to claim 6, wherein the predetermined position in the direction intersecting the vertical direction is a position corresponding to a magnetic field center of the magnetic resonance imaging apparatus.

8. The bed control device according to claim 1, wherein the processing circuitry is further configured to select a support image from among a plurality of original images captured by the imaging device at different timings based on a confidence level set for each original image.

9. The bed control device according to claim 8, wherein the processing circuitry is configured to select an original image with the highest confidence level as the support image.

10. The bed control device according to claim 8, wherein the processing circuitry is configured to select a new original image over time as the support image when there are a plurality of original images having the same confidence level or confidence levels within a predetermined range.

11. The bed control device according to claim 8, wherein the confidence level is a confidence level regarding a projector position on the subject.

12. The bed control device according to claim 8, wherein the confidence level is calculated based on results of skeletal detection of the subject.

13. A bed control device comprising processing circuitry configured to:
calculate a position of a part of a subject to be imaged based on information relating to the part of the subject placed on a top plate provided in a bed device, which is to be imaged by a medical image diagnostic apparatus, and an image including the top plate and the subject placed on the top plate and captured by an imaging device;
estimate a position of the top plate in a vertical direction;
calculate an amount of movement of the top plate in a direction intersecting the vertical direction based on the calculated position of the part of the subject to be imaged and the estimated position in the vertical direction;
control the movement of the top plate in the direction intersecting the vertical direction based on the calculated amount of movement; and
perform scene analysis to analyze imaging scenes captured as a moving image in a continuous sequence by the imaging device, detect a detectable timing, and identify a frame image corresponding to a frame of the moving image extracted at the detectable timing as a support image.

14. The bed control device according to claim 13, wherein the processing circuitry is configured to calculate the amount of movement of the top plate in the direction intersecting the vertical direction based on a distance between the position of the part of the subject to be imaged in the direction intersecting the vertical direction and a predetermined position in the direction intersecting the vertical direction.

15. The bed control device according to claim 14, wherein the predetermined position in the direction intersecting the vertical direction is a position within an imaging space in a bore of a magnetic resonance imaging apparatus as the medical image diagnostic apparatus.

16. The bed control device according to claim 15, wherein the predetermined position in the direction intersecting the vertical direction is a position corresponding to a magnetic field center in the magnetic resonance imaging apparatus.

17. The bed control device according to claim 1, wherein the imaging device is an optical camera.

18. A bed device comprising:
the bed control device according to claim 1;
the top plate; and
a bed drive device that moves the top plate in the vertical direction and in the direction intersecting the vertical direction.

19. A magnetic resonance imaging apparatus comprising:
the bed device according to claim 18; and
a gantry in which a static magnetic field magnet for generating a static magnetic field is provided.

20. A bed control method, using a computer, comprising:
calculating a position of a part of a subject to be imaged based on information regarding the part of the subject placed on a top plate provided in a bed device, which is to be imaged by a medical image diagnostic apparatus, and an image including the top plate and the subject placed on the top plate and selected from a plurality of original images captured by an imaging device at different timings based on a confidence level set for each original image;
estimating a position of the top plate in a vertical direction;
calculating an amount of movement of the top plate in a direction intersecting the vertical direction based on the calculated position and the estimated position; and
controlling the movement of the top plate in the direction intersecting the vertical direction based on the calculated amount of movement.
